(19) United States
(12) Patent Application Publication
(10) Pub. No.: US 2022/0000493 A1
(43) Pub. Date: Jan. 6, 2022

(54) Endplate Processor

(71) Applicants: Zongde YANG, SHANGHAI (CN); Zhicai SHI, SHANGHAI (CN)

(72) Inventors: Zongde YANG, SHANGHAI (CN); Zhicai SHI, SHANGHAI (CN)

(21) Appl. No.: 17/290,718

(22) PCT Filed: Nov. 5, 2018
(86) PCT No.: PCT/CN2018/113984

§ 371 Date: Apr. 30, 2021

(51) Int. Cl. A61B 17/16

(52) U.S. Cl. CPC A61B 17/1659; A61B 17/1671

(57) ABSTRACT

An endplate processor, comprising a grip portion (1), a handle (2), and a treating head (3). The treating head (3) and the grip portion (1) are fixed at both ends of the handle (2), respectively. A semicircular groove (4) is formed on an end face of the end of the treating head (3) away from the handle (2). The semicircular groove (4) forms a bowl-shaped cavity at the tip of the treating head (3). The bowl-shaped structure is suitable for scraping an endplate (20) in the intervertebral space that is wide at the center and narrow at the periphery. Since the volume of the scraping structure is small, the endplate processor can freely move in the intervertebral space, increasing operation convenience. Since the distal end of the endplate processor is blunt due to the bowl shape, it is even harder to penetrate through the aorta deep in the contralateral annulus, increasing the surgical safety.

### BACKGROUND OF THE PRESENT INVENTION

### Field of Invention

The present invention relates to a medical instrument, in particular, to a device for clearing cartilaginous endplates during spine surgeries.

### Description of Related Arts

In spine surgery, bone grafting in intervertebral space is needed after removal of a bulging intervertebral disc (implantation of an autogenous or allogeneic bone graft), such that spinal fusion between two vertebrae occurs after the surgery to ensure stability of a whole spine and prevent adjacent vertebrae from regressing. The post-operative effect of spinal fusion greatly relies on the spinal fusion effect, while the latter depends on the preparation of intervertebral space for bone grafting to a large extent, which means whether or not a cartilaginous endplate below a vertebral plate is completely scraped away to ensure full contact between a vertebral bone face and a graft. Existing surgical instruments for endplate removal are oblong reamers. Since the oblong shape of the reamer does not fit the anatomical shape of endplate clearance that is wide at the center and narrow at the periphery, the effect of endplate removal is usually poor, which is adversely affects the post-operative spinal fusion. FIG. 1 shows a traditional reamer 30 used in prior art. The traditional reamer 30 has a head portion with an oblong structure.

Ar entering a vertebra 10, the traditional reamer fails to touch an endplate 20 in the center area due to its oblong structure, so that the endplate in the center area cannot be completely removed and the cortical bone surface below the endplate cannot be completely exposed, such that the finally implanted graft fails to fully touch the cortical bone, which is adversely affects post-operative spinal fusion.

### SUMMARY OF THE PRESENT INVENTION

To solve problems in the prior art, the present invention provides an endplate processor. A scrapping device at the tail end of the endplate processor is a small and blunt bowl-shaped structure, particularly suitable for scrapping an endplate in the intervertebral space that is wide at the center and narrow at the periphery. Since the volume of the scraping structure is small, the endplate processor can freely move in the intervertebral space, increasing operating convenience. Since a distal end of the endplate processor is blunt due to the bowl shape, it is even harder to penetrate through the aorta deep in the contralateral annulus, increasing surgical safety.

To achieve the above objective, the present invention adopts the following technical solution:

An endplate processor, comprises a grip portion, a handle and a treating head. The treating head and the grip portion are fixed at both ends of the handle, respectively. A semicircular groove is formed on an end face of the end of the treating head away from the handle. The semicircular groove forms a bowl-shaped cavity at a tip of the treating head.

The treating head is a conical frustum. One end of the conical frustum with a smaller diameter is fixedly connected to the handle and one end of the conical frustum with a larger diameter is provided away from the handle. The semicircular groove is provided at the end face of the end with a larger diameter.

Preferably, the conical frustum has a height of 10 mm.

Preferably, the grip portion and the handle are both cylindrical and the diameter of the grip portion is greater than that of the handle.

Preferably, the handle has a diameter of 3 mm and a length of 190 mm; and the grip portion has a diameter of 10 mm and a length of 100 mm.

One side of the semicircular groove toward the handle is recessed.

Preferably, the semicircular groove has a diameter of 5 mm.

Preferably, the handle and the treating head are made of a metal material.

Preferably, the grip portion is made of a wooden material.

Preferably, the metal material is brass.

The present invention has the following beneficial effects: The scrapping device at the tail end of the endplate processor is a small and blunt bowl-shaped structure, particularly suitable for scrapping an endplate in the intervertebral space that is wide at the center and narrow at the periphery; since the volume of the scraping structure is small, the endplate processor can freely move in the intervertebral space, increasing operating convenience; and since the distal end of the endplate processor is blunt due to the bowl shape, it is even harder to penetrate through the aorta deep in the contralateral annulus, increasing surgical safety.

### BRIEF DESCRIPTION OF THE DRAWINGS

The following describes the present invention in further detail in conjunction with accompanying drawings.

FIG. 1 is a schematic diagram of the operating state of a traditional reamer scrapping an endplate according to the prior art;

FIG. 2 is a structural diagram of an endplate processor according to the present invention;

FIG. 3 is a schematic diagram of the operating state of an endplate processor scrapping an endplate according to the present invention;

FIG. 4 is a schematic diagram of the operating state of an endplate processor scrapping an intervertebral endplate according to the present invention.

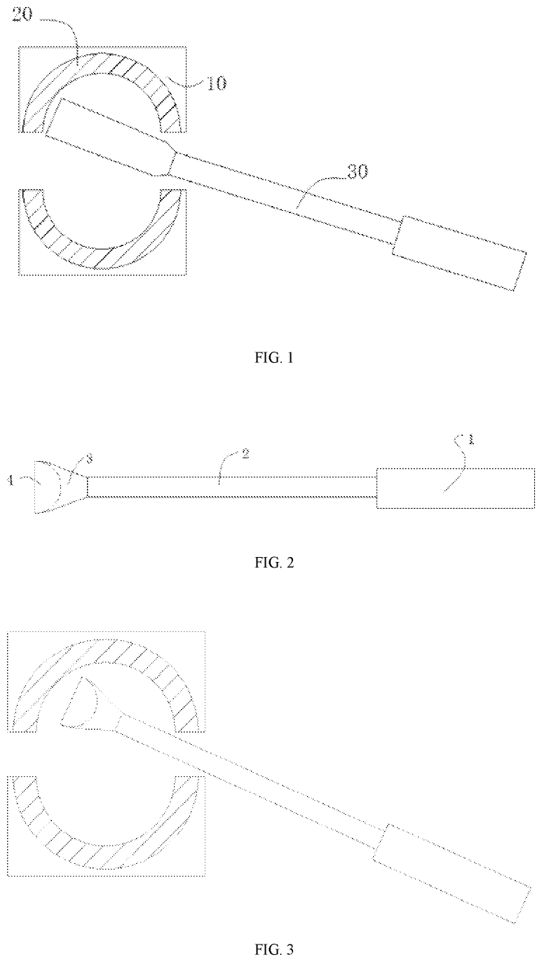

### DETAILED DESCRIPTION OF THE PREFERRED EMBODIMENT

The following describes the present invention in further detail in conjunction with accompanying drawings and specific mode of implementation.

### Embodiment 1

As shown in FIG. 2, an endplate processor comprises a grip portion 1, a handle 2 and a treating head 3. The treating head 3 and the grip portion 1 are fixed at both ends of the handle 2, respectively. The grip portion 1 and the handle 2 are both cylindrical. The diameter of the grip portion 1 is greater than that of the handle 2. The handle 2 has a diameter of 3 mm and a length of 190 mm; and the grip portion has a diameter of 10 mm and a length of 100 mm.

The treating head 3 is provided at an end portion of the handle 2. The treating head 3 is a conical frustum. One end of the conical frustum with a smaller diameter is fixedly connected to the handle 2, and one end of the conical frustum with a larger diameter is provided away from the handle 2. A semicircular groove 4 is formed on an end face of the end of the treating head 3 away from the handle 2. One side of semicircular groove 4 toward the handle 2 is recessed. The semicircular groove 4 forms a bowl-shaped cavity on an end face of the treating head 3. The bowl-shaped cavity is configured to process an endplate. To ensure that the endplate processor has highly accurate operability, the diameter of the semicircular groove 4 is 5 mm. The conical frustum has a height of 10 mm.

The handle 2 and the treating head 3 are made of a metal material, preferably brass; and the grip portion 1 is made of a wooden material.

When needing to process the cartilage of the endplate, a surgeon holds the grip portion 1 and pushes the endplate processor to drive the treating head 3 to move on the surface of the cartilage, such that an edge of the bowl-shaped cavity can scrape the cartilaginous endplate. As shown in FIG. 3, the endplate processor is used at the step of endplate scrapping after complete removal of an intervertebral disc. The treating head 3 with the bowl-shaped structure extends into the intervertebral space. By changing the handle direction, endplate tissues in the central area that cannot be scrapped using the traditional reamer can be removed. After the endplate processor enters the intervertebral space that is wide at the center and narrow at the periphery, it can be seen that the structure of the central area is opposite to the bowl-shaped structure of the endplate, so that the endplate in the central area (which is also a bowl-shaped structure) of the intervertebral space can be easily scraped.

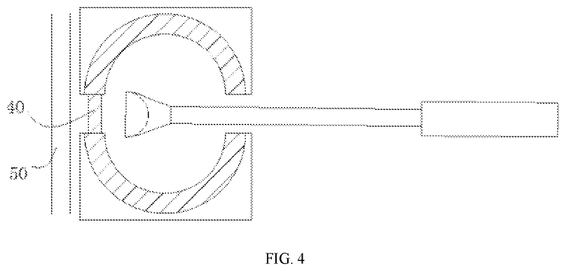

From FIG. 4 it can be seen that the most dangerous area of the intervertebral space is the rear aortaventralis 50. Once the endplate processor penetrates through the annulus fibrosus 40 and reaches the front of the vertebra, it is very easy to damage the aorta, causing danger to the life of a patient. The traditional reamer is an oblong structure, as shown in the figure. The head portion and the intervertebral space happen to be consistent in the oblong shape, so that it is possible that the traditional oblong reamer reaches the front of the vertebra during an operation. The head diameter of the treating head with the bowl-shaped cavity that is utilized by the present invention is larger than the width of the traditional reamer, so that it is very hard to penetrate through the contralateral intervertebral space and the blunt bowl-shaped structure of the endplate processor further ensures protection against damage to blood vessels. Moreover, a round working area is usually formed when the intervertebral space is exposed for vertebral plate removal and there are very sensitive nerve roots on two sides of the round working area, so that it is possible that the traditional reamer stimulates the nerve roots in the process of entering the intervertebral space due to its long oblong structure, causing neurological symptoms to the patient after the surgery. The endplate processor with a bowl-structured head portion that has a smaller diameter passes through the working area easily to enter the intervertebral space without touching the nerve roots on two sides.

Those skilled in the art will recognize that, on the premise of without departing from the protective scope of the present invention, the above mode of implementation can be modified, changed and combined in various ways and will hold the idea that such modifications, changes and combinations are made with an inventive step.

What is claimed is:

1. An endplate processor, comprising a grip portion, a handle and a treating head, wherein the treating head and the grip portion are fixed at both ends of the handle, respectively; a semicircular groove is formed on an end face of an end of the treating head away from the handle; and the semicircular groove forms a bowl-shaped cavity at a tip of the treating head.

2. The endplate processor according to claim 1, wherein the treating head is a conical frustum; one end of the conical frustum with a smaller diameter is fixedly connected to the handle, while one end of the conical frustum with a larger diameter is provided away from the handle; and the semicircular groove is provided at an end face of the end with a larger diameter.

3. The endplate processor according to claim 2, wherein the conical frustum has a height of 10 mm.

4. The endplate processor according to claim 1, wherein the grip portion and the handle are both cylindrical and the diameter of the grip portion is greater than that of the handle.

5. The endplate processor according to claim 4, wherein the handle has a diameter of 3 mm and a length of 190 mm; and the grip portion has a diameter of 10 mm and a length of 100 mm.

6. The endplate processor according to claim 1, wherein one side of the semicircular groove toward the handle is recessed.

7. The endplate processor according to claim 1, wherein the semicircular groove has a diameter of 5 mm.

8. The endplate processor according to claim 1, wherein the handle and the treating head are made of a metal material.

9. The endplate processor according to claim 1, wherein the grip portion is made of a wooden material.

10. The endplate processor according to claim 9, wherein the metal material is brass.
